(19) United States
(12) Patent Application Publication
(10) Pub. No.: US 2007/0284241 A1
(43) Pub. Date: Dec. 13, 2007

(54) Managing A Chemical Reaction And Moving Small Particles

(76) Inventor: Osman Kibar, San Diego, CA (US)

Correspondence Address: FISH & RICHARDSON PC, P.O. BOX 1022, MINNEAPOLIS, MN, 55440-1022, US

(21) Appl. No.: 11/758,492

(22) Filed: Jun. 5, 2007

Related U.S. Application Data
(60) Provisional application No. 60/812,195, filed on Jun. 9, 2006.
(60) Provisional application No. 60/829,820, filed on Oct. 17, 2006.
(60) Provisional application No. 60/860,762, filed on Nov. 22, 2006.

(51) Int. Cl. C07C 1/00; B01J 19/08

(52) U.S. Cl. 204/157.15; 422/186

(57) ABSTRACT

Among other things, a force field is used to manage an aspect of an energy profile of a chemical reaction. In some cases, the aspect of the energy profile is managed to alter the profile or to monitor the profile.

Among other things, an electromagnetic beam and one or more magnetic fields are applied in a controlled manner to manipulate a small particle to move from one location to another based on a magnetic state of the particle.

This application is entitled to the benefit of the filing dates of U.S. provisional applications Ser. Nos. 60/812,195, MANAGING A CHEMICAL REACTION, filed Jun. 9, 2006, and 60/829,820, MOVING SMALL PARTICLES, filed Oct. 17, 2006, and 60/860,762, MANAGING A CHEMICAL REACTION AND MOVING SMALL PARTICLES, filed Nov. 22, 2006, all incorporated here by reference in their entireties.

### BACKGROUND

This description relates to managing a chemical reaction and to moving small particles.

Catalysis, for example, includes processes that increase the rate of a chemical reaction. Catalysts include chemical substances that modify and increase the rate of a chemical reaction without being consumed in the process.

Among different types of catalysis are homogenous catalysis, in which the catalyst and the reactants are in the same phase (e.g., everything in gas phase or everything in a single liquid phase). In heterogenous catalysis, the catalyst and the reactants are in different phases (e.g., catalyst in solid form and reactants in gas or liquid phase; or both catalyst and reactants in liquid form but not dissolved in each other). In autocatalysis, the reaction is catalyzed by one of its products.

In a typical chemical reaction, the reactants will react spontaneously (i.e., without requiring any energy from the outside) to form products if the products have a lower free energy than the reactants (i.e., the reaction has a negative Gibbs energy, ΔG₀<0). FIG. 1 shows energy levels in a chemical reaction. In FIG. 1, the x-axis 102 represents a reaction coordinate and the y-axis 104 represents free energy. E_{A}, that is, activation energy, is an energy barrier that the reactants need to overcome. ΔG₀ represents the energy difference between the reactants 106 and the products 108.

In certain reactions, even though the Gibbs energy is negative, because of E_{A}, the speed of the reaction may become extremely low and practically halt (i.e., the reaction may not occur). In these reactions, the reaction rate typically is proportional to the term exp(−E_{A}/kT), where k is Boltzmann's constant and T is temperature in Kelvin. In other words, the reaction rate slows exponentially based on the ratio of the activation energy to the thermal energy. Catalysis increases the exponential term (towards unity) and thus speeds up the reaction that would have occurred anyway but at a very slow rate. Catalysis affects the speed of the reaction rate towards the steady-state equilibrium concentrations of the reactants and products but ultimately does not change these concentrations.

Two general approaches currently used for achieving catalysis, which may be used separately or in tandem, are thermal (i.e., increase the temperature, T) or chemical (i.e., use a catalyst to effectively reduce the activation energy barrier, E_{A}).

In one thermal method, the temperature of the sample may be increased uniformly, for example, by increasing the temperature of the whole medium. The magnitude of the temperature increase may be limited in practice, especially for biological samples, because most biological molecules of interest (e.g., proteins, enzymes) de-nature (i.e., break up or unfold, and lose functionality) outside a temperature range (˜5-10 degrees above their natural environment).

Another thermal approach irradiates the sample using a wide-band, non-resonant electromagnetic beam in the microwave range. The sample absorbs energy from this beam in such a way that the kinetic energy of polar liquids (e.g., water) is preferentially increased. The solution may be overheated by up to about 10-20 degrees above its boiling point without triggering formation of bubbles (which would have occurred due to boiling). Using this approach, increases in reaction rates of 1 to 2 orders of magnitude have been reported in the literature for certain reactions.

The chemical approach typically uses catalyst molecules (for example, enzymes) or surfaces. Enzymes use chemical mechanisms to manipulate a reaction's energy requirements and effectively reduce the activation energy barrier to achieve catalysis. These mechanisms may be categorized according to how they modify the energy requirements of the reaction.

For example, the ground state energy 210 maybe increased (i.e., reactants are destabilized) by approximation (i.e., proximity) of reactants or by conformational distortion (FIG. 2A).

In approximation, the Gibbs energy is given as ΔG=ΔG₀+RT ln([products]/[reactants]), where R is a constant, square brackets [ ] refer to concentrations, and both terms on the right hand side are negative quantities for a typical reaction. When the enzyme binds to the reactants, it fixes their relative motion and orientation with respect to each other, increasing their effective concentrations (with respect to their concentrations in solution), effectively raising the ground state energy to make the Gibbs energy more negative (i.e., more favorable reaction towards the products) and reducing the activation energy barrier.

In conformational distortions, the enzyme binds to the reactant and changes its conformation such that the environment is now less favorable to the reactants. The reactants' effective ground state energy is similarly increased and the effective E_{A} is reduced.

In other examples, the energy of an intermediate state 310 (FIG. 2B) is stabilized, that is, a local minimum is either created or made more stable (i.e., the energy well is made deeper) in the energy diagram, e.g., by covalent catalysis or by general acid-base catalysis. When the enzyme creates a favorable environment for the intermediate state 310 (i.e., energetically stabilizes it), the activation energy barrier is effectively reduced to that of the largest remaining step. That is, if E_{A} is split into E_{A1}+E_{A2}, then the reaction rate is dominated by the term exp(−E_{A1}/kT) (assuming E_{A1}>E_{A2}), instead of by exp(−E_{A}/kT). The gain in reaction speed is exponential, i.e., by a factor of ˜exp[(E_{A}-E_{A1})/kT].

In some cases, the energy of the transition state 410 (FIG. 2C), which constitutes the peak of the activation energy barrier, may be decreased (i.e., transition state is stabilized), e.g., using preorganization of the active site for transition state complementarity. E_{A} is the reduced activation energy for the transition state 410. In this case, the enzyme's active site is prearranged to complement the transition state (rather than the ground state reactants), which decreases the effective energy of the transition state, and thus, decreases the magnitude of the activation energy barrier.

Besides catalysis, it is of great interest to manipulate the motion (rather than the reaction energetics) of small particles (e.g., molecules, cells, etc.) and there exist many electric and/or magnetic force based techniques to do so.

In electrophoresis (http://en.wikipedia.org/wiki/Electrophoresis), one applies a constant (i.e. non-oscillating) electric field to move an electrically charged particle from one point to another point (i.e., linear motion). In this case, the applied force on the particle of interest is Lorentz force: F=q E, where F is the force acting on the particle, q is the particle's charge, E is the applied electric field, and bold letters imply vectors. In this technique, the particle's motion is opposed by a friction force, which depend on the particle's characteristics (e.g. size, shape, viscosity). The difference between the applied and the opposing forces may then be used to move or separate certain particles from others (e.g. as in gel electrophoresis, http://en.wikipedia.org/wiki/Gel_electrophoresis).

A similar technique is magnetophoresis, where a constant (non-oscillating) magnetic field is applied to interact with a particle's magnetic susceptibility.

Some techniques use an AC field (i.e., oscillating) to generate a driving force on the particles, e.g. dielectrophoresis, optophoresis, or laser tweezers. In dielectrophoresis (http://en.wikipedia.org/wiki/Dielectrophoresis), an oscillating electric field interacts with the electric dipoles of the particle, which lowers the potential energy of the system, in turn creating a force on the particle to move it towards the point of maximum electric field intensity (such that the system energy may be minimized). The force acting on the particle is equal to the negative gradient of the potential energy: F(r)=−∂U(r)/∂r, and U(r)=−p·E(r), where p is the electric dipole moment, E(r) is the applied electric field as a function of the physical dimension r, and U(r) is the potential energy of the particle at that position. The magnitude of the applied force on the particle is proportional to the difference between the dielectric constants of the particle and of the background medium.

Optophoresis (of which Kibar is the inventor) relies on a similar force, except that the applied field is at optical frequencies, so the electric dipoles of the particle that interact with the optical beam are those that can oscillate at these higher frequencies (e.g. electron clouds, rather than heavy ions).

In both dielectrophoresis and optophoresis, the applied force is opposed by a friction force (dependent on the particle's characteristics), and the balance of these two forces is used to move, separate, and sort out particles of interest.

In all of the above cases, the random Brownian motion (due to thermal noise) lowers the resolving power (or specificity) of the technique. That is, particles of similar properties (e.g. size or charge) cannot be distinguished from one another, and thus, cannot be sorted out reliably. And if the application is to classify these particles, similar issues arise in readout resolution and error rates. In addition, there is the issue of particle size. For smaller particles (e.g. small molecules, peptides, proteins), Brownian motion planks a bigger role (since its magnitude relative to the applied external force increases), and the manipulation of the particle becomes even noisier.

Furthermore, there is the issue of available quantity for the particles. If one wishes only to classify the particles (e.g. as in, gel electrophoresis) rather than separate them for further use, the results are limited by the low quantities available for many particles (e.g., low abuhundanc proteins). And for bigger particles (e.g., cells), the forces required to move them require much higher energies to be applied, which increases the noise and lowers the resolving capability of the system (i.e., lower output purity and/or lower yield).

### SUMMARY

In a general aspect, an aspect of a chemical reaction is managed by manipulating energy levels of reactants of the chemical reaction by applying a force field to at least one of the reactants.

Implementations may include one or more of the following features. The force field includes an electromagnetic field, or an oscillating electric or magnetic field. The frequency of the force field is slightly offset from a characteristic resonant frequency of at least one of the reactants. The electromagnetic field includes a non-constant intensity field in time. The electromagnetic field includes a non-constant intensity field in space. The electromagnetic field includes a constant intensity field. At least one of the reactants is in a ground state. At least one of the reactants is in an intermediate state. At least one of the reactants is in a transition state. The aspect of the chemical reaction includes the speed of the chemical reaction. The speed of the chemical reaction is increased or decreased. The aspect of the chemical reaction includes a final composition of the chemical reaction. The chemical reaction includes a catalytic process. The manipulating includes using a modified spectroscopy technique. An energy level of a ground state of the reactants is increased or decreased. An energy level of a transition state is decreased or increased. An intermediate state is stabilized or de-stabilized. The spectroscopic technique includes interaction of the force field with quantized energy levels of the reactants. The spectroscopic technique includes magnetic resonance spectroscopy. The spectroscopic technique includes electron spin resonance. The spectroscopic technique includes nuclear magnetic resonance. The spectroscopic technique includes rotational spectroscopy. More than one spectroscopic technique is used in tandem. A mechanism decouples resonance. The mechanism includes applying radiation at a resonant absorption frequency to saturate a particular energy resonance to decouple that resonance from other resonances. The mechanism includes an inversion recovery sequence. The reactants are subjected to the electromagnetic beam at a frequency selected to cause an anomalous dispersion effect of the index of refraction to generate differential potential energies along a configuration space. The intensity of the electromagnetic beam is changed to control the speed of the chemical reaction in a continuous way. The electromagnetic beam is applied for a pre-determined period of time before being turned off or is applied for a period of time that is determined by feedback obtained from the system. A particular chemical bond type in the chemical reaction is targeted by the electromagnetic beam. The specificity of a particular interaction between at least two reactants is managed. More than one electromagnetic beam is used at different frequencies targeting more than one resonance. More than one reaction is managed, including simultaneously and in the same solution, sequentially and in the same solution, or simultaneously and in separate solutions. More than one electromagnetic beam is used at different frequencies, with at least one of the frequencies not resonant with the reactants. The reactants are subjected to a separate electromagnetic beam, its frequency being selected to cause absorption of energy by at least one of the reactants, and the resulting absorption profile is used to measure the composition of the reactants at a particular time during the chemical reaction. The separate beam is applied while the first beam is still on or after it is turned off. At least one of the reactants is in liquid phase. At least one of the reactants is in gas phase. The chemical reaction includes in vivo reactions. The chemical reaction includes in vitro reactions. The temperature of the sample is controlled. A second aspect of the chemical reaction is controlled. More than one chemical reaction is controlled.

In general, in an aspect, spectroscopic techniques are used to subject reactants of a chemical reaction to an electromagnetic beam having a frequency. The frequency of the electromagnetic beam is adjusted to sweep through a desired range of spectrum such that an aspect of the chemical reaction is changed at a particular frequency.

Implementations may include one or more of the following features. The change in the aspect includes an increase or decrease in the speed of the chemical reaction. The change in the aspect includes a change in the final composition of the chemical reaction. The particular frequency is monitored. An electromagnetic beam is applied at the particular frequency to manage the aspect of the chemical reaction.

In general, in an aspect, an apparatus includes a device to establish a force field, a reactor for a chemical reaction, and a controller to manipulate energy levels of reactants of the chemical reaction to control an aspect of the chemical reaction.

Implementations may include one or more of the following features. The reactants reside in a fixed solution or they flow through the region of applied field. The reactor includes a container or a chip. The reactor is compartmentalized to separate reactants or chemical reactions. The reactor has input/output interfaces. The reactor is connected to a sample preparation unit or an output extraction unit. The apparatus is not portable or it is portable. The controller has a software module. The controller uses at least one database. The controller controls reaction parameters such as temperature or pH. The controller is controlled by a human operator or it is automated.

In general, in an aspect, a force field is used to manage an aspect of an energy profile of chemical reaction. In some implementations, the aspect of the energy profile is managed to alter the profile or to monitor the profile.

Implementations may include one or more of the following features. The electromagnetic beam is circularly polarized. One reactant is an enantiomer or a chiral molecule. The differential potential energy is generated at or around a conical intersection.

In general, in an aspect, an electromagnetic beam and one or more magnetic fields are applied in a controlled manner to manipulate a small particle to move from one location to another based on a magnetic state of the particle.

Implementations may include one or more of the following features, and other features. The magnetic state comprises a spin state of the particle (e.g., an electron spin state or a nuclear spin state) induced by an applied magnetic field. Small particles in the one location may have other magnetic states that are not caused by the applied electromagnetic beam and magnetic fields to move from the one location to the other location. The small particle may be a molecule. The small particle may be one of a set of small particles having a common magnetic state and the electromagnetic beam and the magnetic fields may be applied to separate all of the set of small particles from other particles in the one location that do not share the common magnetic state. The particle may have a particular magnetic state associated with its molecular structure . The applied magnetic field is constant and uniform. The magnitude of one of the magnetic fields is significantly smaller than the magnitude of a second of the magnetic fields. At least one of the applied magnetic fields has a controlled spatial profile. At least one of the applied magnetic fields has a controlled temporal profile. The magnitude of at least one of the magnetic fields is caused to vary spatially over time to cause a continuous relocation of the small particle toward a desired location. The intensity of the electromagnetic field has a controlled spatial profile. The frequency of the electromagnetic field has a controlled spatial profile. The small particle comprises an enantiomer. The electromagnetic field is circularly polarized.

Other aspects include other combinations of these and other aspects and features expressed as methods, apparatus, systems, and program products, and in other ways.

Other advantages and features will become apparent from the following description and the claims.

### DESCRIPTION OF DRAWINGS

FIGS. 1 and 2A through 2C are energy diagrams.

FIGS. 3A through 3C are graphs.

FIG. 4 is a block diagram.

FIG. 5 is a schematic diagram.

FIGS. 6 through 9 are graphs.

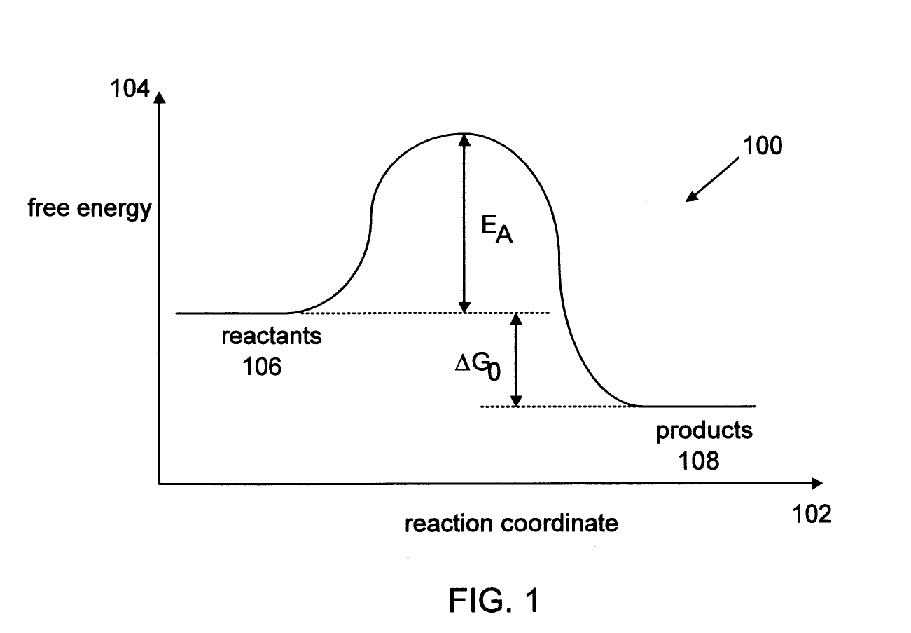

### DETAILED DESCRIPTION

Energy level manipulations in chemical reactions can be achieved by mechanisms in addition to thermal or chemical, for example, mechanisms that depend on force fields (e.g., oscillating electric, magnetic, and/or electromagnetic fields, and in particular, resonant fields).

In some examples of spectroscopic measurement, one probes quantized energy levels of a sample using an electromagnetic beam and uses the resulting absorption (or emission) spectra to deduce the properties of the sample. Various spectroscopy techniques are suitable to study various types of samples and/or phenomena.

For example, far ultraviolet x-ray beams may be used to study the ionization or dissociation of molecules, and visible and UV light may be used to probe electronic state transitions. Near-infrared beams are useful to study vibrational states of a sample (from which fundamental vibrational frequencies and force constants can be derived). Rotational spectroscopy (in the microwave range) is used to study the rotational spectra (especially of gas phases, where the rotational motion is quantized), which yields information about moments of inertia, interatomic distances, and angles.

In magnetic resonance spectroscopy, a DC magnetic field (i.e. constant and non-oscillating) is applied to provide energy level separations probed by the radiation (otherwise, these levels are degenerate, and thus, have the same energy). Nuclear magnetic resonance (NMR) studies isotopes of elements having net nuclear spin (e.g., hydrogen) to provide high resolution information on bond distances and orientation. Because the nuclei have small magnetic moments, the probe radiation falls in the radio-frequency range. Electron spin resonance (ESR) (or electron paramagnetic resonance) applies to a sample that has a net electron spin (e.g., free radicals, odd-electron molecules, triplet states of organic molecules, and paramagnetic transition metal ions and their complexes). The required probe frequencies fall in the microwave range due to the much higher magnetic moment of an unpaired electron (compared to a nuclei's magnetic moment).

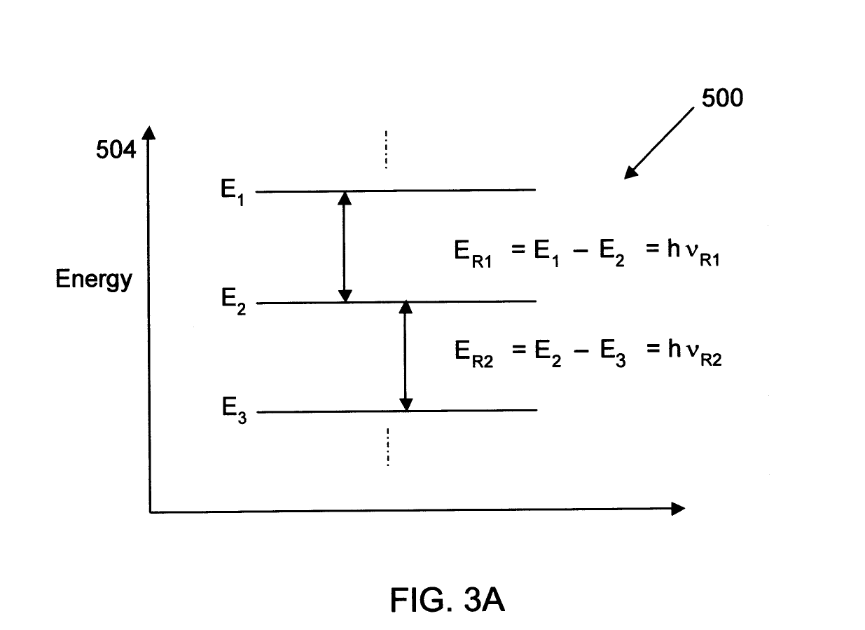

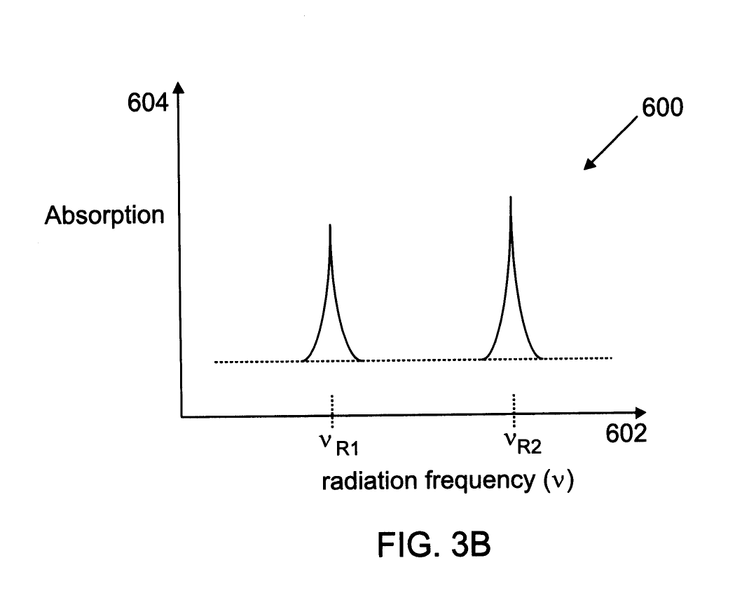

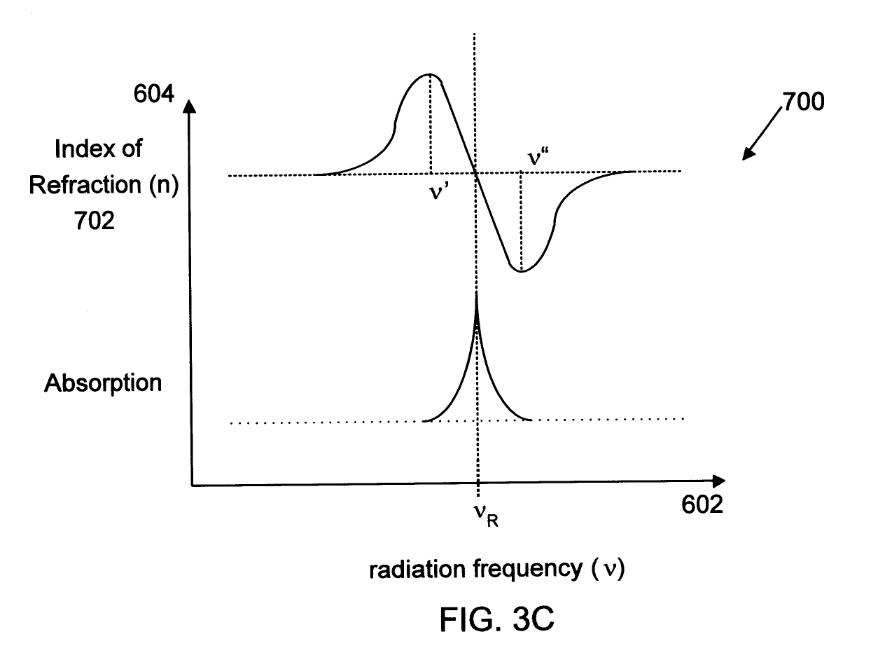

A simple picture for each technique is that the sample has many characteristic energy levels as shown in FIG. 3A. The y-axis 504 in FIG. 3A represents energy. E₁, E₂, E₃ are the different energy levels. E_{R1} represents the energy difference between E₁ and E₂, and E_{R2} represents the energy difference between E₂ and E₃. ν_{R1} and ν_{R2} are the frequencies corresponding to E_{R1} and E_{R2} respectively. For NMR or ESR, these levels are induced by applying a constant external magnetic field. For each technique and for each sample, an electromagnetic beam's energy is swept through an appropriate range of frequencies. Whenever the radiation frequency matches that of a characteristic resonant frequency of the sample, the sample absorbs energy from the beam (provided, of course, that the selection rules are satisfied). An absorption peak that is centered around that resonant frequency is observed.

In FIG. 3B, the x-axis 602 represents radiation frequency and the y-axis 604 represents absorption. FIG. 3B shows two absorption peaks with one peak occurring at radiation frequency ν_{R1} and one at radiation frequency ν_{R2}. The energies at which these absorption peaks occur (i.e., resonant frequencies, ν_{R}) and their magnitudes are analyzed to yield information about the properties of the sample.

Due to anomalous dispersion, the index of refraction of the sample around a resonant frequency has the form shown in FIG. 3C, in which the refractive index n (702) is equal to (εᵣ μᵣ)^{1/2}, and εᵣ and μᵣ are the relative permittivity and relative permeability, respectively. From FIG. 3C, the refractive index of the sample increases as the radiation frequency approaches (from below) a resonant frequency peaking at ν′, then decreases along a negative slope, crossing the background index (i.e., the non-resonant index due to normal dispersion) exactly at the resonant frequency at ν_{R}. The refractive index of the sample continues to decrease as the radiation frequency exceeds resonance reaching a minimum at ν″. After that, it increases to its background value. An index of refraction represents an aggregate effect and references to refractive index of a single molecule should be interpreted in a qualitative way.

The energy of a system is modified when the sample is under an externally applied field. The potential energy of a molecule with an electric moment (p) or with a magnetic moment (μ) is given as: U=−p·E, or U=−μ·B, where U is the potential energy, E and B are the applied electric and magnetic fields, respectively. The bold letters imply that the variables are vectors, and · is the dot product of two vectors. The electric/magnetic moments may be permanent, or they may be induced by an external field.

These equations imply that externally applied fields will create additional potential energy components for molecules with electric/magnetic moments (permanent or induced). A force will be exerted on the molecules to shift them into a state where this potential energy is minimized (i.e., most negative).

For example, in the dielectrophoresis technique, an external electric field having a spatial component is applied, so that E becomes a function of x (i.e., E becomes E(x)), where x signifies any one of the three spatial dimensions. The potential energy becomes a function of physical space, i.e., U becomes U(x)=−p·E(x). Because the (negative) gradient of energy is force (i.e., F(x)=−∂U(x)/∂x), assuming a constant electric moment, a force is now exerted on the molecules of interest to physically move towards the point of maximum E(x) (i.e., where potential energy of the system, U(x), is most negative, and thus, is at a minimum).

In the technique described here, a similar type of force is exerted on the molecules of interest. In some examples, the externally applied field may be constant in x (i.e., E(x)=E everywhere in physical space); however, due to a resonant anomalous dispersion effect that the external field has on the particular molecules of interest, the electric/magnetic moment becomes a function of a configuration space, i.e., p(R) or μ(R), where R signifies a dimension of different chemical reactions, molecular structures, and/or bonds. In other words, points along the R dimension constitute different forms that a molecule may take on.

Under the influence of a resonant external field, the system experiences a force along configuration space (i.e., F(R)=−∂U(R)/∂R), because potential energy is now a function of configuration space, and thus, is not constant for all configurations. In other words, the externally applied (uniform) field exerts a force on the system to minimize its potential energy, such that the system now favors certain reactions, bond formations, and molecular structures over others (i.e., it favors certain states along the configuration space). The result is that the energy landscape of the system may be manipulated, and the energy levels of certain bonds and molecules may be shifted (as described below).

To apply this technique to catalysis, we modify the setup of spectroscopic techniques and apply external fields to exert similar forces (i.e., arising from different indices of refraction, which translate to different electric/magnetic moments) on molecules and reactions and bond formations, such that the catalyst functionality is achieved without having to use thermal or chemical means.

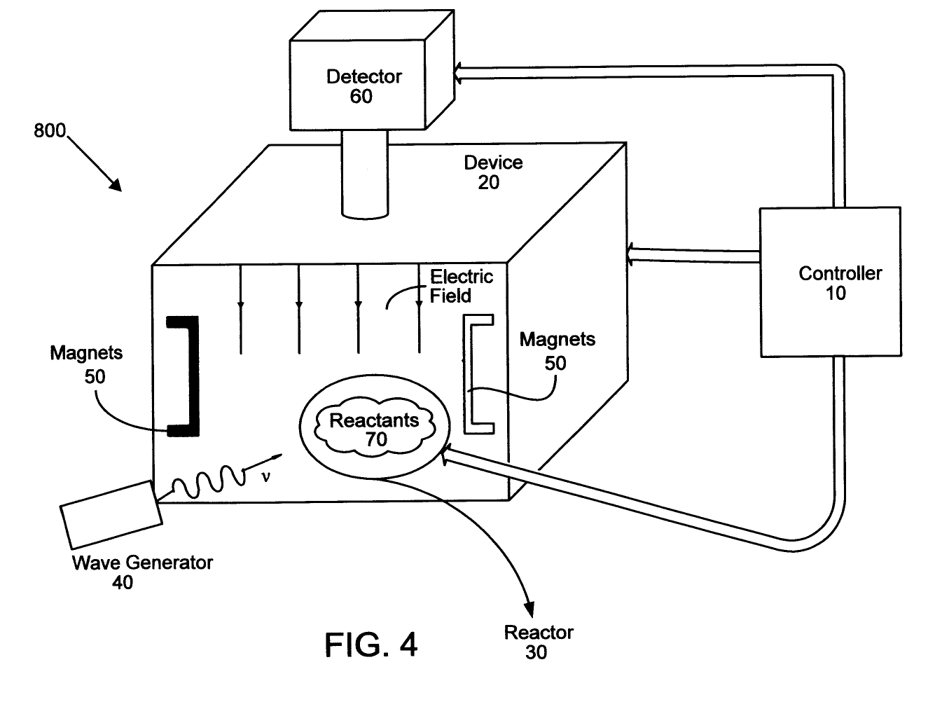

FIG. 4 demonstrates how the setup of a spectroscopic technique can be adapted to manage a chemical reaction. A controller 10 controls and coordinates a device 20, a detector 60, and a reactor 30. The controller 10 directs the device 20 to turn on or off an electric field and to activate or deactivate magnets 50. The controller 10 also commands a wave generator 40, which is part of the device 20, to generate electromagnetic waves. The frequency of the electromagnetic wave, ν, may be adjustable. Reactants 70 are contained inside the reactor 30. The reactor 30 may be a container, or a chip, or a compartmentalized device that can be used to separate reactants or chemical reactions or for other purposes. A detector 60 is used to monitor the chemical reaction. It may also send feedback to the controller 10.

In some examples, e.g., when the spectroscopic technique is ESR, the wave generator 40 in FIG. 4 represents a microwave-generating Klystron tube. The detector 60 represents a diode detector. In an ESR experiment, samples are mounted into a microwave cavity. The reactor 30 in FIG. 4 represents the microwave cavity. Other equipment used in an ESR experiment, such as an attenuator or a circulator, is collectively represented by the device 20.

In some examples, we start with a particular spectroscopic technique (e.g., NMR) and a particular molecule of interest that is susceptible to the chosen type of spectroscopy (e.g., molecules containing hydrogen, which have a net nuclear spin). The frequencies of resonance of the hydrogen molecule depend on the types of atoms that lie within a certain number of bond lengths of the hydrogen molecule (e.g., current NMR techniques can differentiate up to 3 bond lengths).

For example, when a hydrogen atom makes a bond with another atom, such as nitrogen or carbon, the particular bond will influence the electron cloud around the hydrogen nucleus. Then, the types of atoms and bonds that this group of atoms has (e.g., —NH₂ or NH₃, C—CH₂ or CH₃) will cause an additional chemical shift on the hydrogen's spectra, so the resonant frequencies of hydrogen will slightly shift to other frequencies. Therefore, each characteristic resonance will have a slightly different ν_{R} (and a corresponding ν′ and ν″ as in FIG. 3C) based on the chemical environment that this hydrogen nucleus experiences.

In spectroscopy, a certain range of frequencies of electromagnetic radiation is applied. The resulting absorption spectra implies a list of resonant frequencies (i.e., a list of ν_{R}) and their magnitudes. Analyzing all such peaks and their magnitudes enables deductions about molecular structures and bonds and bond distances.

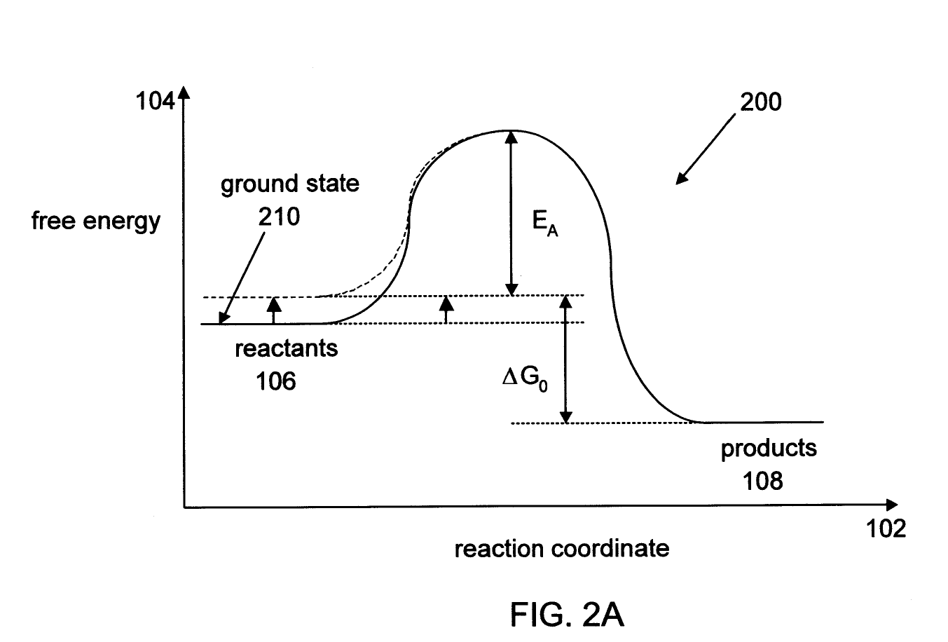

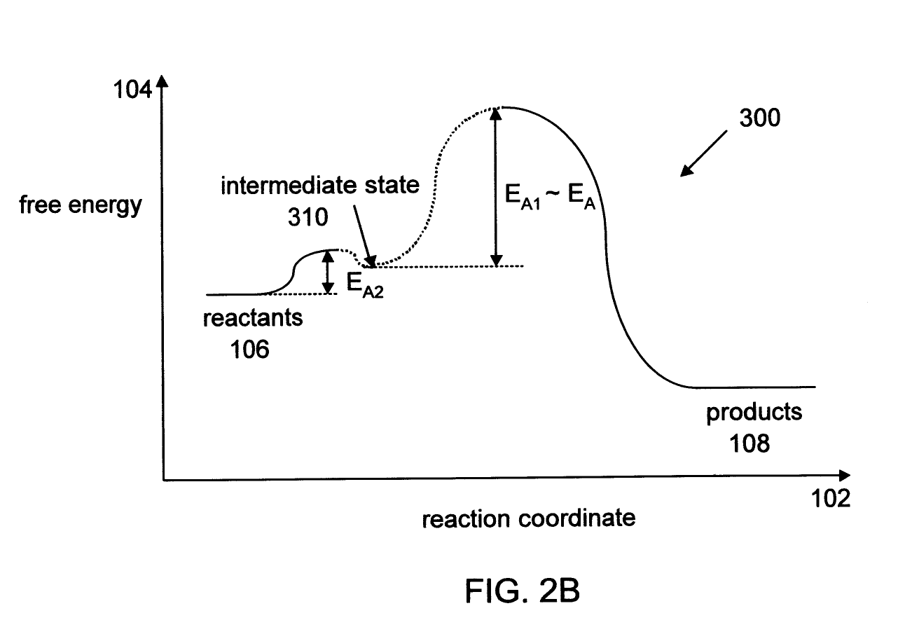

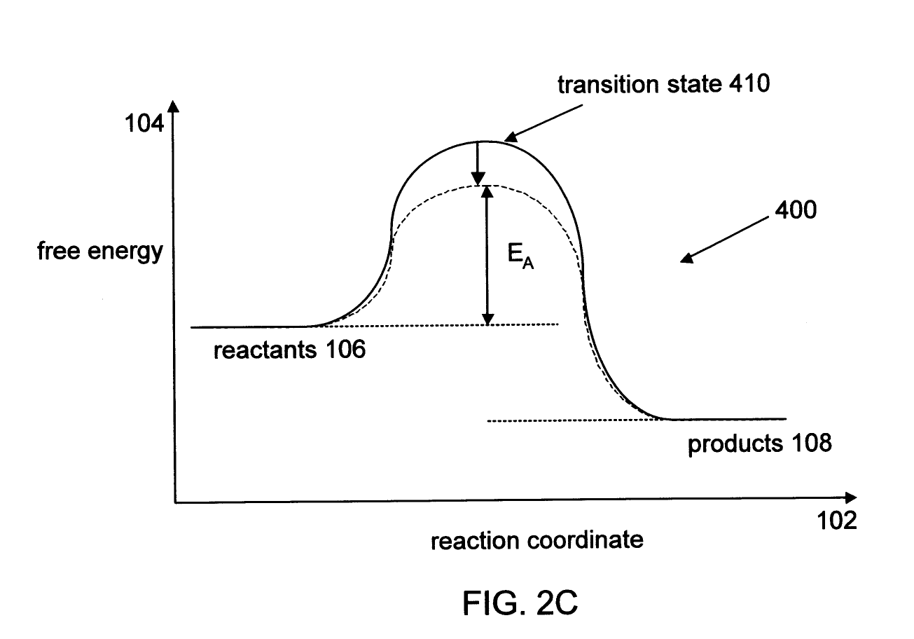

In some examples, resonant frequencies are known for a particular molecule, and an electromagnetic beam is applied to the sample at a frequency that is slightly offset from ν_{R}. If the molecule comprises a transition state of a chemical reaction (as in FIG. 2C), then we set the applied frequency to ν′, where the molecule's effective index of refraction (due to this resonance) is higher than its background index. In this case, the applied external field creates an environment for this molecule having a lower potential energy (i.e., more negative) than before. In other words, the transition state experiences a decrease in its potential energy when the external field is present. The increase in refractive index due to a resonance is relatively sharp (along the frequency axis), so such a potential energy decrease is not experienced by other molecules (which do not have resonances at the same frequency). Therefore, this transition state is stabilized (relative to the system without the external field and relative to the ground state or the product state). As a result, the applied external field effectively lowers the free energy of the transition state and catalyzes the reaction (as shown in FIG. 2C).

Similarly, to achieve the manipulation described in FIG. 2A (i.e., to increase the free energy of the ground state), that ground state's resonant frequencies are determined first. Then, an electromagnetic radiation at the corresponding frequency ν″ is applied. Because the index of refraction of the ground state molecule decreases at this frequency, the potential energy that it experiences under such an external field increases (with respect to the rest of the system). Therefore, the ground state is said to be de-stabilized.

The same scheme may be used to target stabilization of an intermediate state (as in FIG. 2B). For example, assume the intermediate state in question requires the formation of a particular bond, which would not have formed due to energy considerations if the system were left on its own. We would calculate the resonant frequencies (which would have existed if the repulsive energies could be somehow overcome to form this bond) and apply an external electromagnetic frequency at the corresponding ν′. Then, the force that arises from this decrease in potential energy of the system may be used to overcome such repulsive energies and allow the desired bond to be formed even if stable only transitionally. And if this bond already existed (i.e., there is already a local energy minimum in the diagram, such that the intermediate state is already a real state, as opposed to a virtual state), then the force may be used to increase the stability of the bond (i.e., increase the depth of the local minimum). Furthermore, even if the drop in the intermediate state's potential energy (due to the applied force) is not indeed sufficient to create a stable state (i.e. a local minimum in the energy landscape, with zero derivative along the configuration axis, is not created), the time duration of the virtual state may still be extended and its relative stability may be increased. In either case, stabilization of such an intermediate state, in turn, would achieve the catalysis shown in FIG. 2B.

The three schemes mentioned above (to lower the effective activation energy of a reaction) all refer to relative changes in potential energy. That is, when an external field is turned on, even the molecules that do not have a resonance around that frequency may experience a potential energy change due to their permanent (or induced) electric or magnetic moments, which constitute the index of refraction due to normal dispersion (i.e., due to non-resonant fields). Therefore, the various frequencies of radiation (i.e., ν′ and/or ν″) need to be selected appropriately such that the energy levels will be shifted as desired relative to the energy levels in the remainder of the system.

Other spectroscopic techniques may be used for catalysis. For example, if the molecule to be targeted is a free radical, a modified ESR setup can be used, because ESR is applicable to free radicals (i.e., molecules with unpaired electrons). Because ESR operates in a microwave region, the forces in question would be much higher than those used in NMR (which fall in the RF range). Other spectroscopic techniques based on quantized energy levels as mentioned above may also be used under similar conditions.

In some examples, the reactants may be in gas phase or in liquid phase. The reactants may also interact with particles or surfaces that are in solid phase.

In some examples, more than one resonance may be targeted for a particular setup, e.g., by applying multiple radiation beams, each one at an appropriate but different frequency. More than one (modified) spectroscopy technique may also be used in combination at the same time or in sequence. For example, NMR and ESR may be used together (e.g., ENDOR—electronic nuclear double resonance). In this case, a magnet may be used for both techniques to separate the energy levels of interest. Then, two beams of radiation may be applied simultaneously, one in the microwave and the other in the radio-frequency range.

In some examples, because the molecule does not absorb energy from the radiation (or absorbs very little) during the manipulation of potential energy (i.e., no absorption, but only resonant field coupling through index of refraction), one may use an oscillating electric field or an oscillating magnetic field to induce catalysis (instead of an electromagnetic field).

In some examples, radiation at a certain resonant frequency (i.e., at ν_{R}) may be applied to saturate a particular energy resonance and thus de-couple it from other resonances. For example, in hydrogen NMR, the resonance of water (H₂O) may be saturated and thus de-coupled from other resonances of interest, so as to enhance the sharpness and magnitude of these other resonances. In some examples, other similar methods that are routinely used in spectroscopy to decouple certain resonances may also be used here, e.g. inversion recovery sequence.

In some examples, the magnitude of the applied external field may be increased to exert a stronger force on the molecules towards catalysis. The intensity of the field dictates the probability of the molecules of interest interacting with the field, but not whether the above mentioned effects will experience resonance or not. As such, the applied field's intensity does not necessarily have to be uniform across the sample (unless a readout of the reaction status is desired). Due to this additional degree of freedom, larger or different shape containers for the solutions and reactions may be used to achieve higher throughput of products or other desired results.

In some examples, one may decrease the temperature to achieve a higher depth for the intermediate state's local energy well (because the depth of the local energy minimum will increase relative to thermal energy, kT, and thus, a higher stability for this intermediate state).

In some examples, one or more of these modified spectroscopy techniques may be used in tandem (simultaneously or in sequence) with chemical and/or thermal methods (mentioned in the background section) to enhance catalysis. For example, when used in combination with a chemical catalysis technique (e.g., with enzymes present in the solution), one may achieve the same catalytic performance, but with much better catalyst stability (e.g., better thermal range, lower degradation of catalysts, higher robustness to a changing/varying environment, and/or higher mechanical flexibility). Similarly, when used in combination with thermal methods (e.g., microwave heating), our technique, in some examples, may improve the performance of the overall system such that the need for toxic or flammable solvents is eliminated, or reactions may be halted before undesired by-products are output.

In some examples, one or more of the applied fields may be at non-resonant frequencies, in which case, a detailed study of the various reaction states (i.e., ground, intermediate, transition, or product states) and their background index of refraction may be used to identify certain reactions and conditions where catalysis will still be achieved due to shifting of their respective potential energy levels relative to each other (resulting in similar changes in energy diagrams shown in FIG. 2). In other words, the potential energy shifts may arise from the interaction of the external (non-resonant) field with the index of refraction of the various molecules due to normal dispersion (as opposed to anomalous dispersion), and a relative shift in the potential energies of certain states with respect to that of other states may still lead to catalysis.

Unlike the spectroscopy tools from which this technique is derived, this technique does not require any read-out modules (unless they are desired to monitor performance or the concentrations of reactants and products). Some examples may use stripped-down spectroscopy equipment to reduce cost. Other cost-reducing modifications may also be made, e.g., elimination of modules and components that are used to smooth out the applied field intensity and make it more uniform across the sample.

In some examples, the reactants may reside in a fixed solution, or may be flowed through the region of applied field. The reactants may be placed in a container, or positioned on a chip. There may be different containers for reactants, intermediates, products, and/or by-products (i.e., the system may be compartmentalized). There may also be separate storage places for each.

In some examples, commercial equipment may be chosen to optimize the performance of the particular application, e.g., different magnet size (for magnetic resonance), different bands or tunability (adjustable frequency) for the microwave source (e.g., Klystron), etc.

In some examples, typical techniques that are used in spectroscopy to enhance the coupling of the external fields with the energy levels of the molecules of interest may also be used. For example, in microwave spectroscopy for rotational spectra of gases, one may reduce the pressure of the gas to reduce the broadening of energy levels.

In some examples, the apparatus may be fixed or portable. The process may also be controlled by software. Databases and/or lookup tables (e.g., of various parameters and values of reactions) may be incorporated into the management of the technique and the apparatus. Certain control parameters (e.g., temperature, pH, magnitude and frequency of applied fields) may be controlled by an operator. These parameters may be fixed for a given setup, or they may be adjustable by the operator.

In some examples, input/output (I/O) interfaces may be integrated. There may be sample preparation steps (for input reactants), output extraction steps (e.g., separate chambers, automated extraction), and/or interconnection between such steps (e.g., fluidic channels).

In some examples, the change in potential energy can be controlled by adjusting the magnitude of the external field and/or the applied frequency, thus, the magnitude of the force acting on the target molecule (for stabilization or destabilization). This can be used to control and adjust the chemical reaction rates in a continuous manner.

In some examples, the applied electromagnetic beam may be circularly or elliptically polarized (as opposed to linearly polarized or unpolarized), such that a particular enantiomer or a chiral molecule is stabilized (or destabilized). The resulting technique may be used in the preferential management of chiral synthesis processes (e.g. in asymmetric synthesis).

By using the above three schemes in reverse, a particular reaction can be retarded. For example, instead of applying a frequency at ν′, we can apply it at the corresponding ν″ (or vice versa) to move the energy levels in the opposite direction to those given in FIG. 2 (e.g., energy of the transition state may be increased in FIG. 2C, etc).

Catalysis of a reaction may be turned on or off at will at a particular time, manually (e.g., allow the speed up of the reaction rate for a particular period of time) or based on feedback from the system (e.g., catalysis may be stopped when the output concentration reaches a certain level).

One may use the techniques to speed up or slow down particular reactions in chemistry that do not necessarily involve catalysis or an activation energy barrier. In other words, the above mentioned manipulation of energy landscapes may be applicable to any chemical reaction and not just the ones involving catalysis with an energy barrier.

In some examples, conical intersections in chemical reactions and pathways (http://en.wikipedia.org/wiki/Conical_intersection) may be managed such that the reaction flow may be preferentially directed towards a particular path over another (that is energetically similar).

In some examples, the speeds of more than one reaction (with different sets of reactants) may be controlled in tandem. These reactions may be run simultaneously (i.e., with the reactants for all the reactions in the same solution), or sequentially (in time), or in parallel (at the same time, but in separate compartments). Any of such reactions (and their corresponding compartments) may be integrated for easy transfer between them. By speeding up or slowing down certain chemical pathways relative to others (in multi-step reactions), one may manipulate the final composition of products.

In some examples, the specificity of a reaction may be controlled or enhanced, by manipulating the interactions of a specific reactant molecule (relative to other molecules). Particular bond types may be targeted (e.g., covalent, hydrogen, ionic, sulfide bonds, prosthetic groups, etc.) for manipulation of their energy levels.

In some examples, this technique may also be used for chemical reactions occurring in vivo (i.e., inside a living organism, e.g., a human, an animal, bacteria).

The above described technique may be used in a variety of applications and industries where catalysis may be of benefit to the overall performance. These include, but are not limited to: medical uses (e.g., medicinal chemistry, biopharmaceuticals, biotechnology, proteins), energy applications (e.g., waste management, power/fuel alcohol, byproduct and biogas, mineral oils and drilling muds), food industry (e.g., potable alcohol, baking, brewing, dairy, flavouring, fruit juice, dextranase and sugar processing, edible oils, glucose oxidase, wine), and other industrial applications (e.g., analytical applications, detergents, colouring, leather, paper, plant tissues, starch, textiles, immobilized enzymes, membrane cleaning, yeast extract). For these applications, the technique may reduce the need (or even eliminate) certain steps that are currently used, e.g., identifying the required catalysts and reaction steps, manufacturing these catalysts, and purifying the catalysts from the products after the reaction has been catalyzed, etc.

In some examples, the technique may be used to emulate the functionality of different kinds of enzymes and other biocatalysts (as listed in the Enzyme Data Bank or other search bases), e.g., oxidoreductases, transferases, hydrolases, lyases, isomerases, and ligases.

Besides inducing catalysis, the described technique may be used to monitor catalytic and/or non-catalytic reactions, e.g., pause during the application of the external field and take a readout of the reactants and/or products (since the setup for this spectroscopy will already be available).

In some examples, the technique may be used to emulate unknown (or un-manufacturable) catalysts, by either constantly sweeping the frequency of the externally applied field, or running this sweep until the frequencies that enable catalysis are identified, and then fixing the applied field at these frequencies. This method may eliminate the need to know the catalytic steps a priori and the need to manufacture the required catalyst molecules or surfaces. The same process of emulation may be applied to unknown (or uncontrollable) chemical pathways (instead of only those reactions that involve catalysis).

The above variation may also be used to discover and analyze previously unknown reactions, by sweeping the frequency of the external field, taking note of at which frequencies catalysis occurs, and then analyzing this data (along with possibly other spectroscopic measurements made with the same setup) to identify these reactions.

All the above examples can be practiced independently or can be combined.

In addition to manipulating the energy landscape of chemical reactions, the technique and the same or a similar force described here may be used to manipulate the motion of small particles (which we call resonant field based spinphoresis, or simply, spinphoresis), in a setup that moves the particles in a selective manner.

Certain particles have a net electron spin (e.g. free radicals, odd-electron molecules, triplet states of organic molecules, and paramagnetic transition metal ions and their complexes). Similarly, certain atoms have a net nuclear spin (e.g. hydrogen). In a paramagnetic material, the constituent atoms or molecules have permanent magnetic dipole moments. However, in the absence of an external magnetic field, the two spin states of each magnetic dipole (i.e. spin up and spin down) are said to be degenerate, that is, their energy levels are practically the same, so the two states are equally occupied and the net magnetization of the material is zero. In magnetic resonance spectroscopy, an external constant magnetic field is applied to separate the energy levels of these two possible spin states. In this case, more electrons will reside in the spin state with the lower energy and the total sum of all spins will be a nonzero value (and hence the paramagnetism, which is induced net magnetism in the presence of an external magnetic field). For a given magnitude of applied magnetic field, the exact energy separation depends on the chemical environment of the unpaired electron. The energy difference between the two spin states are then probed with an electromagnetic beam, and the resulting absorption spectra are analyzed to extract information on the structure and other properties of the molecules of interest. Typically, one can distinguish the surrounding chemical environment up to three bond distances.

For experiments that involve the electron spin, the absorption spectra, and thus the applied electromagnetic frequency, fall in the microwave region. And for ones with nuclear spin, they fall in the RF region. For the rest of this description, we will only refer to the case of electron spin, for simplicity, but the same approach and results also apply to nuclear spin.

For our purposes of manipulation of particles (we use the term manipulation broadly to include any motion of the particles for any purpose including manipulation, separation, and others), we will use a different aspect of the same force. That is, we apply a constant magnetic field and an electromagnetic beam to cause an interaction of the beam with the spin states of the molecule of interest (whose energy levels are separated via the external magnetic field). In other words, the external magnetic field induces a magnetic dipole in the molecule (via its electron spin), which then interacts with the magnetic field of the applied microwave beam. To this end, we describe an example implementation in which this interaction creates a force to move that particle or particles of interest in a different manner than it does other particles. For example, for a set of isomers (i.e. molecules of same composition but different arrangement, http://en.wikipedia.org/wiki/Isomer), only particles of one isomeric arrangement will move in a given direction, while none of the other particles will experience any net force. The applied force will be a function of “molecular structure”, and therefore, will be extremely specific. We will then discuss some possible variations of this technique.

To describe how resonant-field based spinphoresis works, we will provide one example setup in some detail, and then some other variations will be mentioned.

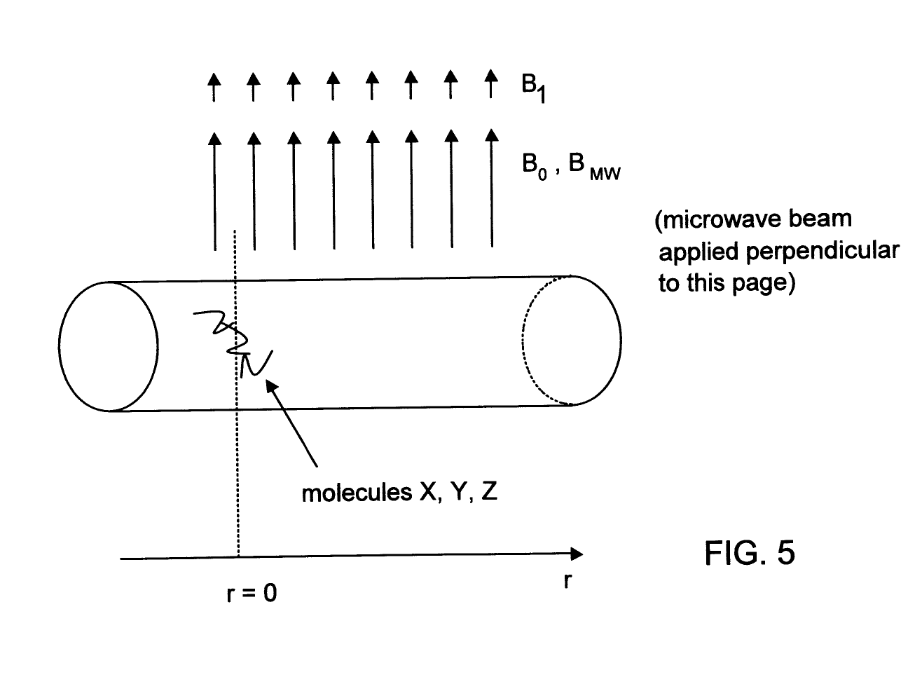

Assume a one-dimensional container (shown as a cylinder in FIG. 5), with three individual molecules of three different types in solution (X, Y, and Z), all of them mixed together and positioned at one point in the container (e.g. at r=0). We intend to separate molecule X from the other two. Further assume that X and Y are paramagnetic molecules, and Z is not. In other words, in the presence of an applied magnetic field, X and Y exhibit an induced net magnetization, and Z does not. X and Y may be two isomers (i.e., with the same chemical equation, same molecular weight, same charge, etc), and their only difference is the arrangement of their atoms (i.e., molecular structure). Paramagnetism is an aggregate effect and we're using it here in the context of a single molecule only for analogy purposes. By that, we imply that a net magnetic dipole moment with non-degenerate spin energy levels may be induced by an external magnetic field Oust as we speak of an optical index when talking about the electric dipole moment of an individual molecule, except that it is the permeability that is of interest here, rather than the permittivity).

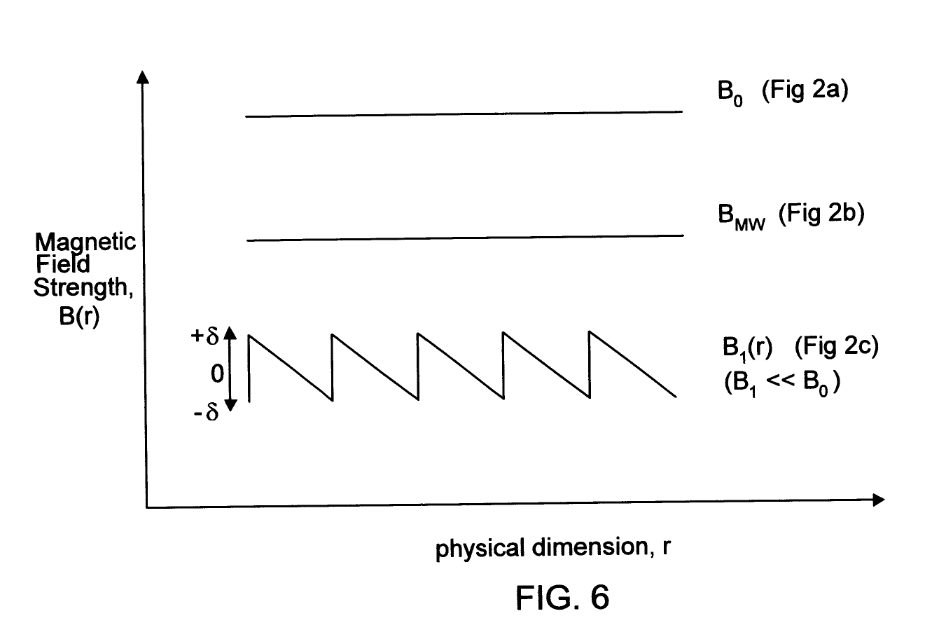

To this container, we apply a uniform constant magnetic field (B₀) (FIG. 6). That is, in this example, it is constant in time (i.e., not oscillating), and it is constant along r: B₀ (r, t)=constant. Since molecule Z is not paramagnetic, B₀ will have no effect on it, whereas the energy levels of the two spin states of both molecule X and of molecule Y will be separated. The energy separation will be: ΔE=gₑμ_{B}B₀, where gₑ is the gyromagnetic ratio of the electron (i.e. ratio of its magnetic dipole moment to its angular momentum) and μ_{B} is the Bohr magneton. However, the exact magnitude of this separation will ever so slightly be different (i.e. ΔE_{X}≠ΔE_{Y}) because of the difference in the chemical environment that the unpaired electrons (of molecules X and Y) see in their respective molecules (i.e. due to spin-orbit coupling). Let's call the corresponding absorption frequencies (i.e. the resonant frequencies) of molecules X and Y: ν_{R-X} and ν_{R-Y}, respectively.

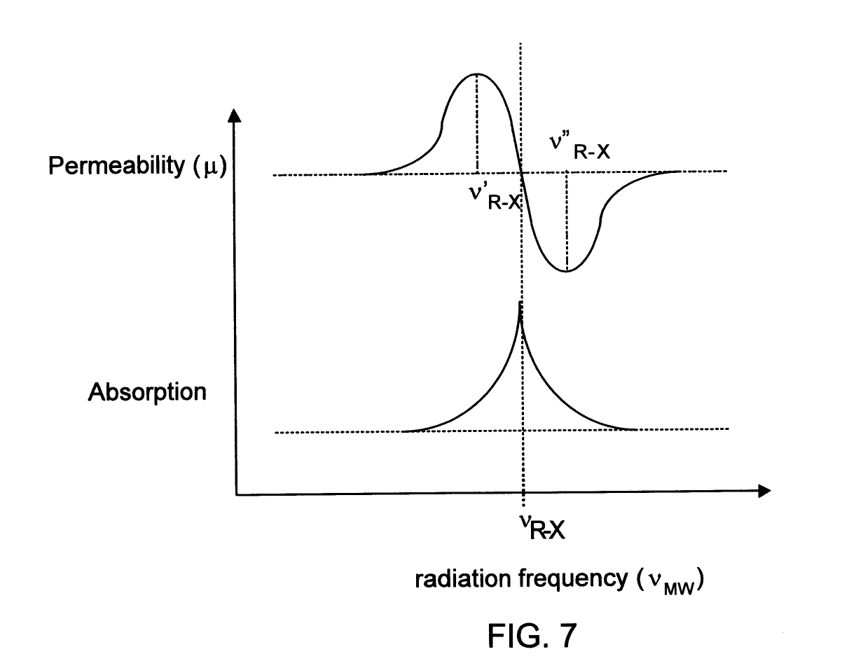

Now, we apply a monochromatic electromagnetic field (in the microwave region), whose frequency equals ν_{R-X} (i.e. ΔE=h ν_{R-X}), so the beam's magnetic field interacts with the molecules via their magnetic permeability (FIG. 7). This beam is oscillating in time, but its intensity is constant along r: B_{MW}(r)=constant, where the MW subscript refers to the magnetic field component of the microwave beam (rather than that of the constant magnetic field, B₀). B₀ and B_{MW} vectors are assumed to be co-linear (the microwave beam may be linearly polarized to ensure this co-linearity).

At this point, the two spin states of molecule X exhibit a resonance with the microwave beam, since the energy separation due to the applied magnetic field exactly matches the photon energy of the microwave beam. Therefore, molecule X will be absorbing energy from this beam, and molecule Y and Z will not (at least, not due to a resonance).

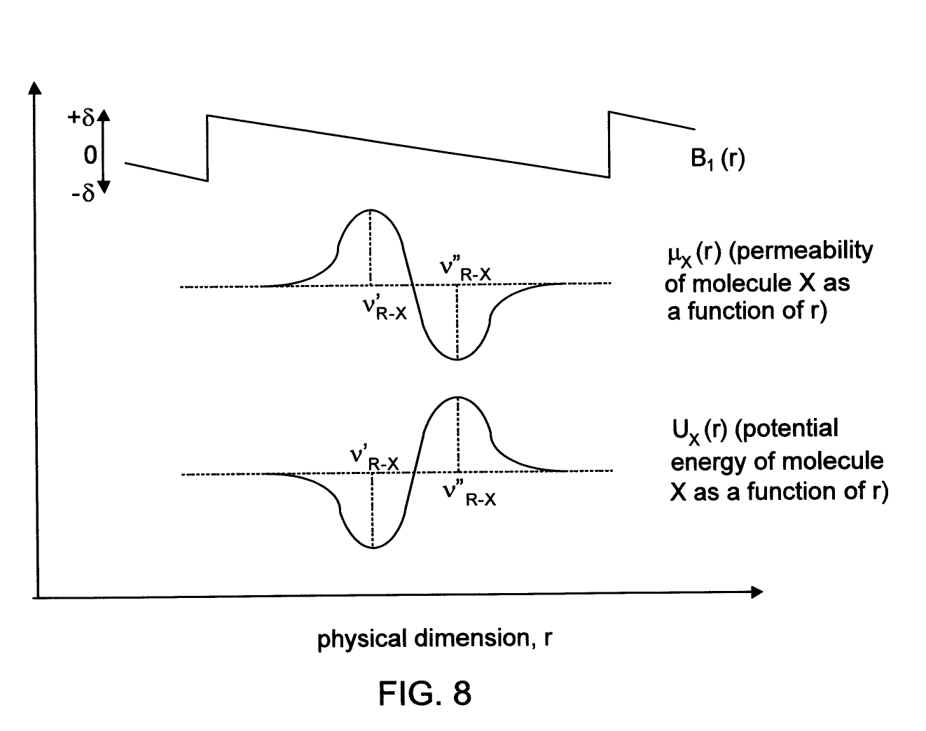

Now, we apply a second external (non-oscillating) magnetic field (B₁). B₁ is in the same direction as B₀ (i.e., parallel), is much smaller than B₀ in magnitude, and is a function of r. For this example, assume B₁(r) is a sawtooth function, centered at 0, and goes from −δ to +δ (FIG. 8) in successive teeth of the sawtooth. As B₁ goes to −δ, the total external magnetic field acting upon the molecule (i.e. that is inducing the magnetic dipole moment) decreases to B₀−δ. Provided the applied microwave frequency stays the same at ν_{R-X}, B_{MW} is now above resonance (because, effectively, the absorption frequency of molecule X has shifted to a lower resonant frequency, ν⁻_{R-X}). Similarly, when B₁ goes to +δ, the total external magnetic field increases to B₀+δ (so the new effective resonant frequency of molecule X shifts to a higher resonant frequency, ν⁺_{R-X}), and the applied microwave beam (with magnetic field constant at B_{MW}) is now below resonance. In other words, for the applied magnetic field B₀, B₁ effectively brings molecule X into and out of resonance with the microwave field B_{MW}.

As a practical matter, the above setup will probably have B₁ go from 0 to 2δ and B₀ will initially be set to a value that is lower by δ. This way, the resulting energy range swept by the two magnetic fields combined will still be the same, and yet the circuitry to generate B₁ will need to flow current in only one direction (because going from a positive to a negative magnetic field strength, one needs to flow current in the opposite direction). However, to illustrate our point in a more intuitive way, we will continue to assume the range of B₁ to be between ±δ (and thus, the total covered range to be between B₀±δ).

Now, let's look at the potential energy of each of the three molecules (residing in a system with the above described fields), and the forces arising from this energy landscape. For a magnetic dipole, the potential energy is given by: U(r)=−μ·B, where U is the potential energy, μ is the magnetic dipole moment of the molecule (which is induced by B₀), and B in this equation is the magnetic field of the microwave beam (B_{MW}). In our setup, B_{MW} is constant in r, but μ is not (because of B₁). As a result, it is more appropriate to write the potential energy equation as follows: U(r)=−μ(r)·B_{MW}.

Molecule Z, remember, is not paramagnetic or magnetic, so it does not have any induced net magnetization. As such, the applied magnetic fields do not change its potential energy, so U_{Z}(r)=constant.

Molecule Y is paramagnetic; however, for an applied magnetic field in the B₀±δrange, its magnetic dipole does not exhibit a resonance with the applied microwave at the ν_{R-X} frequency. As such, its magnetic moment is constant in r, and so is its potential energy: U_{Y}(r)=constant.

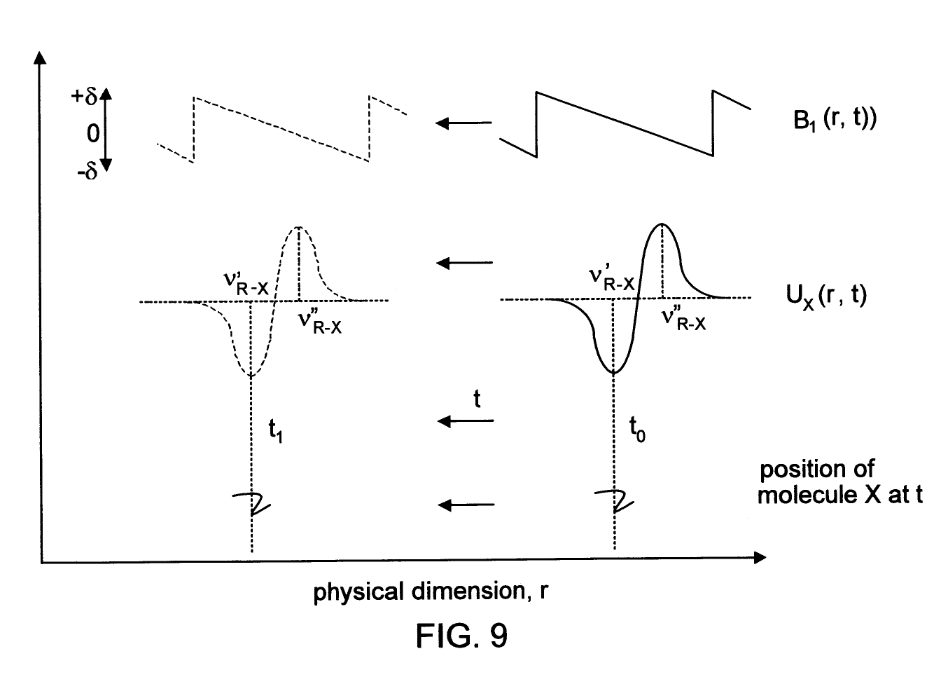

Finally, for molecule X, the story is different. Molecule X is paramagnetic, and it has a resonance with the microwave beam of ν_{R-X} frequency under an applied magnetic field of B₀. From anomalous dispersion, we know that each resonance (i.e. each absorption peak at ν_{R-X}) is accompanied by a positive index peak (implying a higher permeability than its background value) to the left of the absorption peak (i.e. at a slightly lower frequency than ν_{R-X}, labeled as ν′_{R-X}) and a negative index trough (implying a lower permeability than its background value) to the right of the same absorption peak (i.e. at a slightly higher frequency than ν_{R-X}, labeled as ν″_{R-X}) (FIG. 9). This means that at ν′_{R-X}, the induced magnetic dipole moment is stronger, so the molecule interacts more strongly with the microwave beam's magnetic field (B_{MW}), and the potential energy of the system (U_{X}) is lower (FIG. 9). Similarly, at ν″_{R-X}, μ is lower and U_{X} is higher. Therefore, when B₁(r) is applied (assuming δ is selected to be big enough such that the applied range of the total magnetic field covers both ν′_{R-X} and ν″_{R-X}), the induced magnetic dipole moment (and thus, the potential energy) of molecule X becomes a function of r. As B₁(r) goes to +δ, the total magnetic field increases, the applied microwave frequency effectively shifts to below resonance (towards ν′_{R-X}), and potential energy decreases. And the opposite trend is true for B₁ (r) approaching −δ (with the potential energy increasing).

Since force is always given as the negative gradient of energy (i.e. derivative along r), this setup creates a net force (as a function of r) acting on molecule X, with no net force on molecules Y or Z. That is, the applied fields act together to physically move molecule X towards the point where its potential energy is minimized (i.e. where its induced magnetic dipole moment is maximum), whereas they have no effect on other molecules, which do not exhibit a resonance in that range.

Now that we have a net force acting on molecule X (and on no other molecule), we can use it to separate molecule X from all others in a very selective manner. To achieve this specific motion (the motion is linear and we more broadly include any sort of translational motion, whether or not linear, in the term motion, as distinguished from, for example, rotational motion of a particle that remains at a given location), we turn B₁ (r) into a traveling wave (i.e. B₁(r,t)). Imagine the sawtooth function mentioned previously, and now we move that whole function along the −r direction (FIG. 9). As B₁ moves, the point of minimum potential energy for molecule X will shift as well, which will in turn apply a physical force on molecule X to follow and to move along with B₁ (along −r direction), with the point of minimum potential energy (corresponding to ν′_{R-X}) attracting molecule X and the point of maximum potential energy (corresponding to ν″_{R-X}) repelling it (i.e. just like in dielectrophoresis or in optophoresis, where a particle's electric dipole moment follows a moving electric field, dragging the particle with it). As long as the speed with which B₁ moves is at a slow enough pace (such that molecule X can follow despite the opposing friction force), molecule X will physically move. On the other hand, if you recall, this same force does not create a net force for either molecule Y or Z, because their potential energies are constant in r. Therefore, the force, which is the gradient of energy, becomes zero. As a result, those molecules will not move (except for Brownian motion, which on average constitutes a zero net force).

Note that this technique is specific with respect to the molecular structure of the particle, that is, with respect to the chemical environment that the (unpaired) electron sees around it. Any molecule that fits the description (i.e. exhibiting a resonance for the particular magnitudes of the applied magnetic fields and microwave beam) will experience a net (nonzero) force to move in a particular direction, whereas those molecules without a resonance in the applied range are not affected at all. This is different from the other techniques mentioned above, where the separation is achieved by the (typically small) difference in the net force experienced by each molecule (i.e. applied force minus the opposing forces). That is, all molecules with the same characteristic (e.g. all charged molecules) move, except that some move more than others, which inherently limits the resolving capability of the separation procedure.

This technique can be applied to molecules and/or particles that differ with respect to a wide variety of properties, such as size, composition, weight, shape, magnetic susceptibility, charge, chirality, etc. The particles being manipulated may be small molecules, macromolecules, biocomplexes, peptides, proteins, bacteria, cells, nanoparticles, microparticles, quantum dots, etc.

In the example setup described above, the microwave frequency was kept constant and the total applied magnetic field was changed (via B₁). One may also achieve similar results by keeping the magnetic field strength constant, and instead changing the microwave frequency (and thus, its energy).

B₁ may be any of a variety of various different functions (instead of the sawtooth example given above).

B₁ (as well as B₀ or B_{MW}) may be varying in time and/or in space.

The microwave frequency may be selected to be in different bands (i.e. different range of frequencies), targeting different molecular structures and magnetic dipoles.

The microwave beam may be circularly or elliptically polarized (as opposed to linearly polarized or unpolarized), such that a particular enantiomer or a chiral molecule is moved preferentially. The resulting technique may be used towards chiral resolution (http://en.wikipedia.org/wiki/Chiral_resolution).

Multiple microwave beams (in different regimes) may be employed to target more than one resonance of the same molecule at the same time.

B₀, B₁, and B_{MW} may be applied at different orientations than described above.

Any of these fields may be continuous or pulsed, and any of them may be constant or varying in time.

They may be constant or varying in space, along the r dimension and/or along the other two physical dimensions (e.g. for manipulation/separation along 2- or 3-dimensions, or for perpendicular motion of particles during flow).

The range of B₁ (which, when combined with B₀, covers the resonant absorption peak of the molecule of interest) may be selected such that it covers both index peaks (i.e. positive and negative peaks, at ν′_{R-X} and ν″_{R-X}, respectively), only one peak (e.g. only positive peak at ν′_{R-X}), or only a partial segment of one peak (e.g. only rising half of the positive peak, that is, the left half of ν′_{R-X}).

The particles may be residing in gas or in liquid.

Similar forces may be generated with other modalities, e.g. using electromagnetic beams in the RF range in an NMR-like setup (i.e., targeting nuclear spins, rather than electron spins, in a setup similar to ones used in nuclear magnetic resonance spectroscopy). One may also generate similar forces in setups similar to rotational microwave spectroscopy (especially applicable for gases).

The technique may be used on particles with other types of magnetism, e.g., ferromagnets, diamagnets (of course, the associated forces will be of different magnitudes).

The technique may target frequencies that are non-resonant with a particular molecule.

Implementations may be based in a solution or in a microfluidic environment (i.e. on a stable surface or in flow).

The movement and separation may be thresholded (or biased) using another force. For example, a small fluidic flow may be set up in the opposite direction of the spinphoresis force. The molecule of interest would have to have a higher spinphoretic force acting on it to overcome this flow and still move in the desired direction, whereas other molecules would not be able to exceed this threshold, and thus, stay in the same region (plus, their diffusion due to Brownian motion may be reduced or eliminated as well).

This force may be another spinphoretic force, or of a different nature, e.g. fluidic, magnetic, electric, gravitational, frictional, or electromagnetic.

This force (which sets up a threshold and/or biases the motion of the particles of interest) may be constant or it may be varying in time and/or in space.

A gradient may be superimposed along the direction of motion (or perpendicular or at an angle to it). This gradient may be of temperature, pH, viscosity, etc. It may be constant or varying in space and/or in time.

This technique may be used for the purpose of many different functionalities, e.g. manipulation, separation, tweezing, chemical reaction management, etc.

It may be used to identify and/or quantify particles.

It may be used to select a certain range of particles (e.g. based on size, shape, or some other property with a desired narrow range).

It may be used to identify, select, manipulate, and/or manage reaction pathways.

It may be used to tag biological spin probes.

The functionality may be implemented in a continuous manner (along a continuous parameter) or in a discrete manner, e.g. separation of particles into one of a predetermined number of categories (i.e. binary if two, multi-level if more than two).

One may implement a given functionality (e.g. separation) only once or multiple times in the same device or system. For example, same microwave frequency may be employed at more than one compartment of the same system, or multiple microwave beams (at different frequencies) may be used in sequence or in parallel at different sections of the system.

The technique described herein may be integrated in the same apparatus with different functionalities, e.g. same device may do catalysis and separation (where separation may be done before, during, or after catalysis).

This phoretic technique may be used in combination with other phoretic techniques (e.g. with dielectrophoresis or magnetophoresis).

Other implementations are also within the scope of the following claims.

What is claimed is:

1. A method comprising
managing an aspect of a chemical reaction by manipulating energy levels of reactants of the chemical reaction by applying a force field to at least one of the reactants.

2. The method of claim 1 in which the force field comprises an electromagnetic field.

3. The method of claim 1 in which the force field comprises an oscillating electric field.

4. The method of claim 1 in which the force field comprises an oscillating magnetic field.

5. The method of claim 1 in which the frequency of the force field is slightly offset from a characteristic resonant frequency of at least one of the reactants.

6. The method of claim 2 in which the electromagnetic field comprises a non-constant intensity field in time.

7. The method of claim 2 in which the electromagnetic field comprises a non-constant intensity field in space.

8. The method of claim 2 in which the electromagnetic field comprises a constant intensity field.

9. The method of claim 1 in which at least one of the reactants is in a ground state.

10. The method of claim 1 in which at least one of the reactants is in an intermediate state.

11. The method of claim 1 in which at least one of the reactants is in a transition state.

12. The method of claim 1 in which the aspect of the chemical reaction comprises the speed of the chemical reaction.

13. The method of claim 12 in which the speed of the chemical reaction is increased.

14. The method of claim 12 in which the speed of the chemical reaction is decreased.

15. The method of claim 1 in which the aspect of the chemical reaction comprises a final composition of the chemical reaction.

16. The method of claim 1 in which the chemical reaction comprises a catalytic process.

17. The method of claim 1 in which manipulating comprises using a modified spectroscopy technique.

18. The method of claim 17 comprising increasing an energy level of a ground state of the reactants.

19. The method of claim 17 comprising decreasing an energy level of a ground state of the reactants.

20. The method of claim 17 comprising decreasing the energy level of the transition state.

21. The method of claim 17 comprising increasing the energy level of the transition state.

22. The method of claim 17, comprising stabilization of an intermediate state.

23. The method of claim 17, comprising de-stabilization of an intermediate state.

24. The method of claim 17 in which the spectroscopic technique comprises interaction of the force field with quantized energy levels of the reactants.

25. The method of claim 17 in which the spectroscopic technique comprises magnetic resonance spectroscopy.

26. The method of claim 25 in which the spectroscopic technique comprises electron spin resonance.

27. The method of claim 25 in which the spectroscopic technique comprises nuclear magnetic resonance.

28. The method of claim 17 in which the spectroscopic technique comprises rotational spectroscopy.

29. The method of claim 17 in which using a spectroscopic technique comprises using more than one spectroscopy technique in tandem.

30. The method of claim 17 in which using a spectroscopic technique comprises a mechanism to decouple resonance.

31. The method of claim 30 in which the mechanism comprises applying radiation at a resonant absorption frequency to saturate a particular energy resonance to decouple that resonance from other resonances.

32. The method of claim 30 in which the mechanism comprises an inversion recovery sequence.

33. The method of claim 17 in which using a modified spectroscopy technique comprises subjecting the reactants to the electromagnetic beam at a frequency selected to cause an anomalous dispersion effect of the index of refraction to generate differential potential energies along a configuration space.

34. The method of claim 33 comprising increasing an energy level of a ground state to speed up the chemical reaction.

35. The method of claim 33 comprising decreasing an energy level of a ground state to slow down the chemical reaction.

36. The method of claim 33 comprising decreasing the energy level of the transition state to speed up the chemical reaction.

37. The method of claim 33 comprising increasing the energy level of the transition state to slow down the chemical reaction.

38. The method of claim 33 comprising stabilization of an intermediate state to speed up the chemical reaction.

39. The method of claim 33 comprising de-stabilization of an intermediate state to slow down the chemical reaction.

40. The method of claim 33 in which the intensity of the electromagnetic beam is changed to control the speed of the chemical reaction in a continuous way.

41. The method of claim 33 in which the electromagnetic beam is applied for a pre-determined period of time before being turned off.

42. The method of claim 33 in which the electromagnetic beam is applied for a period of time that is determined by feedback obtained from the system.

43. The method of claim 33 in which a particular chemical bond type in the chemical reaction is targeted by the electromagnetic beam.

44. The method of claim 33 in which the specificity of a particular interaction between at least two reactants is managed.

45. The method of claim 33 comprising using more than one electromagnetic beam at different frequencies targeting more than one resonance.

46. The method of claim 33 in which more than one reaction is managed.

47. The method of claim 46 in which more than one reaction is managed simultaneously and in the same solution.

48. The method of claim 46 in which more than one reaction is managed sequentially and in the same solution.

49. The method of claim 46 in which more than one reaction is managed simultaneously and in separate solutions.

50. The method of claim 33 comprising using more than one electromagnetic beam at different frequencies, wherein at least one of the frequencies is not resonant with the reactants.

51. The method of claim 33 in which the reactants are subjected to a separate electromagnetic beam,
the frequency of this beam being selected to cause absorption of energy by at least one of the reactants, and in which
the resulting absorption profile is used to measure the composition of the reactants at a particular time during the chemical reaction.

52. The method of claim 51 in which the separate electromagnetic beam is applied while the first beam is still being applied.

53. The method of claim 51 in which the separate electromagnetic beam is applied after the first beam is turned off.

54. The method of claim 1 in which at least one of the reactants is in liquid phase.

55. The method of claim 1 in which at least one of the reactants is in gas phase.

56. The method of claim 1 in which the chemical reaction comprises in vivo reactions.

57. The method of claim 1 in which the chemical reaction comprises in vitro reactions.

58. The method of claim 1 in which the temperature of the sample is controlled.

59. The method of claim 1 also comprising controlling a second aspect of the chemical reaction.

60. The method of claim 1 also comprising controlling more than one chemical reaction.

61. A method comprising
using spectroscopy techniques to subject reactants of a chemical reaction to an electromagnetic beam having a frequency,
adjusting the frequency of the electromagnetic beam to sweep through a desired range of spectrum such that an aspect of the chemical reaction is changed at a particular frequency.

62. The method of claim 61, in which the change in the aspect comprises an increase in the speed of the chemical reaction.

63. The method of claim 61, in which the change in the aspect comprises a decrease in the speed of the chemical reaction.

64. The method of claim 61, in which the change in the aspect comprises a change in the final composition of the chemical reaction.

65. The method of claim 61 also comprising monitoring the particular frequency.

66. The method of claim 65, in which an electromagnetic beam is applied at the particular frequency to manage the aspect of the chemical reaction.

67. An apparatus comprising
a device to establish a force field, and
a reactor for a chemical reaction, and
a controller to manipulate energy levels of reactants of the chemical reaction to control an aspect of the chemical reaction.

68. The apparatus of claim 67, wherein the reactants reside in a fixed solution.

69. The apparatus of claim 67, wherein the reactants flow through the region of applied field.

70. The apparatus of claim 67, wherein the reactor is a container.

71. The apparatus of claim 67, wherein the reactor is a chip.

72. The apparatus of claim 67, wherein the reactor is compartmentalized to separate reactants or chemical reactions.

73. The apparatus of claim 67, wherein the reactor has input/output interfaces.

74. The apparatus of claim 67, wherein the reactor is connected to a sample preparation unit or an output extraction unit.

75. The apparatus of claim 67, wherein the apparatus is not portable.

76. The apparatus of claim 67, wherein the apparatus is portable.

77. The apparatus of claim 67, wherein the controller has a software module.

78. The apparatus of claim 67, wherein the controller uses at least one database.

79. The apparatus of claim 67, wherein the controller controls reaction parameters such as temperature or pH.

80. The apparatus of claim 67, wherein the controller is controlled by a human operator.

81. The apparatus of claim 67, wherein the controller is automated.

82. A method comprising
using a force field to manage an aspect of an energy profile of a chemical reaction.

83. The method of claim 82 in which the aspect of the energy profile is managed to alter the profile.

84. The method of claim 82 in which the aspect of the energy profile is managed to monitor the profile.

85. The method of claim 2 in which the electromagnetic beam is circularly polarized.

86. The method of claim 33 in which the electromagnetic beam is circularly polarized.

87. The method of claim 1 in which at least one reactant is an enantiomer or a chiral molecule.

88. The method of claim 44 in which at least one reactant is an enantiomer or a chiral molecule.

89. The method of claim 33 in which the differential potential energy is generated at or around a conical intersection.

90. A method comprising
applying an electromagnetic beam and one or more magnetic fields in a controlled manner to manipulate a small particle to move from one location to another based on a magnetic state of the particle.

91. The method of claim 90 in which the magnetic state comprises a spin state of the particle induced by an applied magnetic field.

92. The method of claim 91 in which the spin state comprises an electron spin of the particle.

93. The method of claim 91 in which the spin state comprises a nuclear spin of the particle.

94. The method of claim 90 in which other small particles in the one location having other magnetic states are not manipulated by the applied electromagnetic beam and magnetic fields to move from the one location to the other location.

95. The method of claim 90 in which the small particle comprises a molecule.

96. The method of claim 90 in which the small particle is one of a set of small particles having a common magnetic state and the electromagnetic beam and the magnetic fields are applied to separate all of the set of small particles from other particles in the one location that do not share the common magnetic state.

97. The method of claim 90 in which the particle has a particular magnetic state associated with its molecular structure.

98. The method of claim 91 in which the applied magnetic field is constant and uniform.

99. The method of claim 90 in which the magnitude of one of the magnetic fields is significantly smaller than the magnitude of a second of the magnetic fields.

100. The method of claim 90 in which at least one of the applied magnetic fields has a controlled spatial profile.

101. The method of claim 90 in which at least one of the applied magnetic fields has a controlled temporal profile.

102. The method of claim 100 in which the magnitude of at least one ofthe magnetic fields is caused to vary spatially over time to cause a continuous relocation of the small particle toward a desired location.

103. The method of claim 90 in which the intensity of the electromagnetic field has a controlled spatial profile.

104. The method of claim 90 in which the frequency of the electromagnetic field has a controlled spatial profile.

105. The method of claim 95 in which the small particle comprises an enantiomer.

106. The method of claim 90 in which the electromagnetic field is circularly polarized.
